(19) United States
(12) Patent Application Publication
(10) Pub. No.: US 2021/0403509 A1
(43) Pub. Date: Dec. 30, 2021

(54) S-LAYER VACCINE FUSION PROTEINS AND METHODS OF USE

(71) Applicant: Avalon GloboCare Corp., Freehold, NJ (US)

(72) Inventors: Uwe Sleytr, Vienna (AT); David Jin, Staten Island, NY (US)

(21) Appl. No.: 17/353,965

(22) Filed: Jun. 22, 2021

Related U.S. Application Data
(60) Provisional application No. 63/043,318, filed on Jun. 24, 2020.
(60) Provisional application No. 63/060,225, filed on Aug. 3, 2020.

(51) Int. Cl. C07K 14/005; C07K 14/195; C07K 14/32; A61K 39/295; A61K 9/51

(52) U.S. Cl. CPC C07K 14/005; C07K 14/195; C07K 14/32; A61K 39/295; A61K 2039/70; C07K 2319/00; C12N 2770/20022; C12N 2770/20034; A61K 9/5169

(57) ABSTRACT

Described are S-layer fusion proteins comprising a self-assembling domain of a S-layer protein and a viral spike protein or a fragment thereof, a pharmaceutical composition (such as a vaccine) comprising the S-layer fusion protein, and method of immunizing a patient in need thereof comprising administering the vaccine.

### RELATED APPLICATIONS

This application claims the benefit of U.S. Provisional Application No. 63/043,318, filed on Jun. 24, 2020 and U.S. Provisional Application No. 63/060,225, filed on Aug. 3, 2020. The entire teachings of the above applications are incorporated herein by reference.

### BACKGROUND OF THE INVENTION

Since the COVID-19 outbreak caused by the novel Coronavirus (SARS-CoV-2), there is an imminent need to control its spread, particularly by developing an efficient vaccination. The spike protein on the coronavirus surface has been identified as a potential immunogen and vaccine target.

The spike glycoprotein (referred to herein as the “spike protein”) is a structural feature of the SARS-CoV-2 virus and several other viruses and is responsible for binding of the virus particle to a host cell. Spike proteins include, for example, coronavirus (CoV) spike proteins such as the Middle East Respiratory Syndrome-Coronavirus (MERS-CoV) spike protein (described, for example, in US20190351049; the contents of which are expressly incorporated by reference herein), the SARS-CoV-1 spike protein, and the SARS-CoV-2 spike protein. Other structural proteins of SARS-CoV-2 are the membrane and envelope proteins and nucleic capsid proteins (Zhou et al. (2020), Int J Biol Sci 16(10): 1718-1723; the contents of which are expressly incorporated by reference herein). The spike protein comprises two units, namely the S1 and S2 domains (Id.). Cell fusion is initiated when the spike protein (and more specifically, the receptor-binding domain, or RBD, of the S1 domain) attaches with a receptor on the host cell surface and the viral nucleocapsid is delivered into the host cell for replication. The spike protein of SARS-CoV-2 binds to the angiotensin-converting enzyme (ACE2) receptor on human alveolar cells. It has been reported that the receptor-binding motif (RBM) is the main functional motif in the RBD and comprises region 1 and region 2 that form the interface between the spike protein and the ACE2 receptor (Yi et al. Key residues of the receptor binding motif in the spike protein of SARS-CoV-2 that interact with ACE2 and neutralizing antibodies. Cell Mol Immunol 17, 621-630 (2020); the contents of which are expressly incorporated by reference herein). The RBM is the most variable region of the RBD. For example, there is only about 48% amino acid sequence identity between RBMs from SARS-CoV-1 and SARS-CoV-2 and yet the binding mechanism is the same for both viruses (Id.).

There remains an urgent need in the art for effective coronavirus vaccines and more specifically for vaccines that stimulate an immune response against SARS-CoV-2.

### SUMMARY OF THE INVENTION

Different from most vaccines under development, we propose the production of S-layer-coronavirus spike protein-fusion proteins for use as an immunogenic composition, for example, for intranasal and oral vaccination. One goal is to induce sufficient immunization (immune stimulation) against COVID-19 and prevent the development of a severe disease pattern which frequently is accompanied by organ damaging processes (e.g., “cytokine storm” described, for example, in Ye et al. (2020)). The pathogenesis and treatment of the ‘Cytokine Storm’ in COVID-19. J Infect 80(6): 607-613; the contents of which are expressly incorporated by reference herein. In some embodiments, the treatment will induce at least a mild infection process. It is believed that the development of a mucosal vaccine (for intranasal and oral application, for example) will be less demanding than manufacturing vaccines for intramuscular or subcutaneous applications.

Many pathogens initiate infections at the mucosal surface and therefore, mucosal vaccination, especially through oral or intranasal administration routes, is highly desired for infectious diseases. At least twenty coronaviruses have been identified, including some which may be responsible for developing cold-like symptoms. The invention thus encompasses vaccines useful for inducing an immune response against SARS-CoV-2 as well as other coronaviruses and thus could be used to prevent or reduce the severity of COVID-19 and other coronavirus infections and diseases.

The invention thus encompasses an S-layer fusion protein comprising a self-assembling domain of a S-layer protein and a viral spike protein or a fragment thereof, a pharmaceutical composition (such as a vaccine) comprising the S-layer fusion protein, and method of immunizing a patient in need thereof comprising administering the vaccine. The self-assembling domain of the S-layer protein includes a truncated S-layer protein or polypeptide that retains the ability to self-assemble. The term “S-layer fusion protein” encompasses a fusion protein comprising an S-layer protein self-assembling domain and a spike protein or a fragment thereof, for example, a coronavirus spike protein or a fragment thereof In some embodiments, the fragment is an immunogenic fragment of the viral spike protein. Such fragments can include a fragment comprising the S1 domain, the RBD, and/or the RBM.

The invention also includes a composition comprising a plurality of S-layer fusion proteins, wherein the S-layer fusion protein comprises a self-assembling domain of a S-layer protein and a viral spike protein or a fragment thereof, and wherein the plurality of S-layer fusion proteins form a self-assembled structure, a vaccine comprising an effective amount of the composition, and a method of immunizing a patient comprising administering the vaccine. The self-assembled structure can be a flat sheet, an open cylinder, or a vesicle. Self-assembly of S-layer proteins, for example in solution, has been described in Sleytr et al. (2014). S-layers:

principles and applications. FEMS Microbiol Rev. 38(5): 823-864; the contents of which are expressly incorporated by reference herein. The self-assembled structure, e.g, the flat sheet, cylinder or vesicle, can be a monolayer or double layer, for example.

The invention also includes nanoparticles comprising or coated with S-layer fusion proteins a vaccine comprising an effective amount of the nanoparticle, and a method of immunizing a patient comprising administering the vaccine. For example, recombinant S-layer fusion proteins have been shown to generate inclusion bodies (IB) e.g. in E. coli. Many of these proteins can form stacks of two dimension (2D) lattices in the cell. Aggregates can adhere to mucosal cells and trigger endocytosis, thereby inducing an immune response. The invention thus includes purified IBs comprising an S-layer fusion protein, pharmaceutically acceptable compositions thereof (e.g., vaccines), and method of stimulating an immune response comprising administering an effective amount of the purified D3 or composition thereof to a patient in need thereof.

The invention additionally includes a nanoparticle coated with a plurality of S-layer fusion proteins, wherein the S-layer fusion protein comprises a self-assembling domain of a S-layer protein and a viral spike protein or a fragment thereof, wherein the self-assembling domain is attached to the surface of the nanoparticle, and wherein the plurality of S-layer fusion proteins form a crystalline lattice on the surface of the nanoparticle. The invention additionally includes a pharmaceutical composition (such as a vaccine) comprising the nanoparticle, and a method of immunizing a patient in need thereof comprising administering the vaccine. Nanoparticles comprising S-layer fusion proteins can be prepared using methods known in the art including those described in Sleytr et al. (2014). S-layers: principles and applications. FEMS Microbiol Rev. 38(5): 823-864; the contents of which are expressly incorporated by reference herein. Nanoparticles can be pharmaceutically acceptable and include, for example, lipid vesicles. An exemplary lipid vesicle is a liposome. The invention thus encompasses a liposome coated with a plurality of S-layer fusion proteins, wherein the S-layer fusion protein comprises a self-assembling domain of a S-layer protein and a viral spike protein or a fragment thereof, wherein the self-assembling domain is attached to the surface of the nanoparticle, and wherein the plurality of S-layer fusion proteins form a crystalline lattice (e.g., a two-dimensional crystalline lattice) on the surface of the nanoparticle. The lipid vesicle can optionally encapsulate an active agent (a hydrophilic and/or lipophilic agent depending the specific type of lipid vesicle). In certain aspects, the liposome described herein encapsulates a hydrophilic agent or compound (in the aqueous core of the liposome) and/or a lipophilic agent or compound (in the lipidic shell). The described nanoparticle, e.g., the liposome, can comprise different S-layer fusion proteins; for example, the nanoparticle can be coated with a first population of S-layer fusion proteins and a second population of S-layer fusion proteins, wherein the self-assembling domain and/or the spike protein or a fragment thereof of the first and second populations can be different. In certain aspects, the spike protein or a fragment thereof of the second population is different from that of the first population. For example, the different spike proteins or fragments thereof can be from different coronaviruses and/or isolated from (or have the same amino acid sequence of) different genotypes or serotypes (e.g., different genotypes or serotypes of coronavirus or SARS-CoV-2) as described in more detail below.

In certain aspects, the nanoparticle is further coated with a nucleic acid. The nucleic acid or mRNA can encode an antigen, e.g., a peptide or protein. The nucleic acid (e.g., an mRNA) can encode a viral protein or a fragment thereof. In certain aspects, the nucleic acid (e.g., an mRNA) encodes a spike protein or a fragment thereof. In additional aspects, the nucleic acid (e.g., an mRNA) encodes a coronavirus spike protein, e.g., a SARS-CoV-2 spike protein, or a fragment of any of thereof. In specific aspects, the fragment is an immunogenic fragment. In certain aspects, the nanoparticle is coated with more than one mRNAs encoding one or more different polypeptides. The invention encompasses methods of preparing the nanoparticles described herein comprising attaching a nucleic acid (e.g., an mRNA) to the surface of the nanoparticles before, after, or at the same time as the S-layer proteins or S-layer fusion proteins. The invention additional encompasses a vaccine comprising an effective amount of the nanoparticle further coated with a nucleic acid, and a method of immunizing a patient comprising administering the vaccine. As will be understood, the vaccine can comprise an effective amount of the spike protein or a fragment thereof and an effective amount of a nucleic acid that is effective to induce an immune reaction. The invention also encompasses methods of preparing the nanoparticles described herein comprising attaching a nucleic acid to the surface of the emulsomes before, after, or at the same time as the S-layer proteins and/or S-layer fusion proteins.

The invention also encompasses an isolated inclusion body comprising an S-layer fusion protein as described herein, wherein the inclusion body is in particulate form, a pharmaceutical composition (such as a vaccine) comprising an effective amount of the isolated inclusion body, and a method of immunizing a patient in need thereof comprising administering the vaccine. The S-layer fusion protein can be expressed in E. coli, for example, and accumulated in an inclusion body. The inclusion body can be isolated and lipopolysaccharide (LPS) can be removed. In certain aspects, the inclusion body is freeze-dried to form a powder and can be used for mucosal vaccination.

The vaccines described herein can be administered to a mucosal surface, for example, the vaccines can be administered intranasally or orally. The use of S-layer technologies in vaccines and/or for stimulating an immune response has been described, for example, U.S. Pat. No. 5,043,158 and Sleytr et al. (2014). FEMS Microbiol Rev 38 (2014) 823-864; the contents of each of which are expressly incorporated herein by reference), novel S-layer fusion proteins and vaccines can be developed. Intranasal and oral vaccination strategies have been reviewed, for example, in Wang et al. (2015). Intranasal and oral vaccination with protein-based antigens: advantages, challenges and formulation strategies. Protein & Cell:480-503. The vaccines described herein can be administered to a subject or patient in need thereof for the purpose of immunizing and/or stimulating an immune response in the subject or patient. The invention encompasses a method of immunizing a patient against a coronavirus, comprising administering a vaccine as described herein wherein the spike protein or a fragment thereof is a coronavirus spike protein of immunogenic fragment thereof. The invention also includes a method of immunizing a patient against COVID-19 comprising administering a vaccine as described herein wherein the spike protein or the fragment thereof is a SARS-CoV-2 spike protein or immunogenic fragment thereof In certain preferred aspects, the vaccine is administered intranasally or orally. In certain aspects, a biomimetic virus structure (e.g., a nanoparticle or self-assembled structure as described herein) based on a S-layer-spike fusion protein can impart a mild and long-lasting immunizations (repeated oral/nasal applications) and could have several advantages in comparison to a single injection. In certain aspects, one goal is to provide structures (e.g., the nanoparticles, self-assembled structures, and/or inclusion bodies described herein) which may not necessarily induce complete protection against COVID-19 but provide a sufficient immunization (immune-stimulation) which prevents the development of an organ damaging process (or cytokine storm) and results in a mild infection process. In certain aspects, a suitable dosing regimen comprises injecting a single dose. In some embodiments, a suitable dosing regimen comprises administering multiple doses periodically.

One aspect of the invention includes using S-layer proteins or systems that are from thermophilic organisms (e.g., Geobacillus stearothermophilus) and/or mesophilic (e.g. Lysinibacillus sphaericus). In certain aspects, the S-layer proteins are not from organisms which are part of the human microbiome. Non-limiting examples of S-layer proteins that can be used are described in detail below, and specifically in Table 1.

As discussed above, the invention contemplates liposomes coated with the S-layer-spike-fusion proteins. These coated liposomes are “biomimetic virus-envelopes” resembling a “Trojan horse without warriors.” In certain embodiments, the compositions of the invention do not include virus RNA, or other nucleic acids, which are typically present in vaccines based on inactivated viruses. In some embodiments, the fusion proteins of the invention will trigger an immune-response but will not induce any virus replication. In other aspects, the compositions comprise viral nucleic acid, e.g., viral RNA. S-layer fusion proteins attached on virus-sized liposomes where spikes are exposed in an identical or similar orientation as on intact coronaviruses can trigger specific receptors for endocytosis (uptake) mechanisms. As described above, alternative compositions comprise self-assembled structures and can include structures that are rod shaped or spherical self-assembly structures which resemble rod shaped Bacteria or Cocci.

As described above, the present invention contemplates the use of S-layer fusion proteins comprising a self-assembling domain and a viral spike protein or a fragment thereof. Fragments can comprise, for example, the S1 domain, the receptor binding domain (RBD) and/or the receptor binding motif (RBM) of a spike protein. In certain embodiments, the fragment is an immunogenic fragment. The viral spike protein can be a coronavirus spike protein, such as a coronavirus spike protein having the amino acid sequence of the SARS-CoV-2 spike protein. The invention contemplates vaccines or compositions comprising such S-layer fusion proteins, methods of manufacturing such S-layer fusion proteins and methods of immunizing patients with such S-layer fusion proteins.

The method of immunizing (for example, for immunization against COVID-19 or other coronavirus infections and diseases) can comprise one or more administrations of the vaccine. In certain aspects, the vaccine as described herein is administered more than once. In yet additional aspects, the vaccine as described herein is a mucosal vaccine administered more than once, for example, the vaccine can be administered periodically, e.g., about every 6 months or about every year, or at other time intervals that maintain a sufficient level of antibodies or immunity to prevent or reduce severe disease. In additional aspects, the mucosal vaccine as described herein is administered (one or more times) after one or more subcutaneous or intramuscular vaccinations, for example, after subcutaneous or intramuscular vaccination with a vaccine having a different composition than the mucosal vaccine as described herein. The intramuscular or subcutaneous vaccine can, for example, be a nucleic acid vaccine such as an mRNA vaccine.

Thus, the mucosal vaccine comprising an S-layer fusion protein described herein can be administered one or more subcutaneous or intramuscular vaccinations against SARS-CoV-2. Vaccines for immunization against SARS-CoV-2 are currently under development and include, for example, mRNA-1273 (described, for example, Jackson et al. (2020). An mRNA Vaccine against SARS-CoV-2—Preliminary Report. NEJM DOI: 10.1056/NEJMoa2022483; the contents of which are expressly incorporated by reference herein), the chimpanzee adenovirus-vectored vaccine, ChAdOx1 nCoV-19, expressing the SARS-CoV-2 spike protein (described, for example, in Folegatti et al. (2020). Safety and immunogenicity of the ChAdOx1 nCoV-19 vaccine against SARS-CoV-2: a preliminary report of a phase 1/2, single-blind, randomised controlled trial. The Lancet doi.org/10.1016/50140-6736(20)31604-4; the contents of which are expressly incorporated by reference herein), and a recombinant vaccine comprising residues 319-545 of the S-protein RBD (described, for example, in Yang et al. (2020). A vaccine targeting the RBD of the S protein of SARS-CoV-2 induces protective immunity. Nature doi.org/10.1038/s41586-020-2599-8; the contents of which are expressly incorporated by reference herein). The mucosal vaccine described herein can be administered one or more times after a vaccine having a different composition; for example, the mucosal vaccine can be administered periodically, e.g., every 6 months or every year or other time intervals that maintain a sufficient level of immune response antibodies to prevent or reduce severe disease.

When the mucosal vaccine is administered more than once, the S-layer fusion protein(s) of a subsequent vaccination can comprise a different S-layer protein(s) than the previous vaccination(s), for example, to reduce any immune response against the S-layer protein(s). For example, the S-layer fusion proteins in each of the vaccinations can comprise the same spike protein or a fragment thereof but different S-layer proteins, e.g., from different organisms and/or having a different amino acid sequences (e.g., having a sufficiently low amino acid sequence identity and/or homology so as to reduce any unwanted immune response against the S-layer protein(s)).

### DETAILED DESCRIPTION OF THE INVENTION

A description of preferred embodiments of the invention follows.

The words “a” or “an” are meant to encompass one or more, unless otherwise specified.

A “polypeptide” is a polymer of amino acid residues joined by peptide bonds. The term “polypeptide” includes proteins.

The present invention utilizes bacterial surface layer (S-layer) proteins as a carrier to immobilize viral spike proteins on the surface of a nanoparticle. Crystalline bacterial cell surface layers (S-layers) are monomolecular arrays of protein or glycoproteins that are found as the outermost cell envelope component of many bacteria and archeae forming a uniform protein sheet fully covering the bacterial cell at all stages of growth. Their construction principle is based on a single type of protein or glycoprotein assembling into a highly ordered, porous array. An important property of isolated S-layer proteins is their ability to re-assemble into crystalline lattices on, or in, various materials and supports (including, for example, hydrophobic, hydrophilic, non-conducting, semi-conducting, and conducting surfaces) with the same physico-chemical properties found originally on the cell, thus forming stable uniform crystalline mono- or double layers. S-layer lattices are typically composed of identical species of subunits. They can exhibit oblique, square, or hexagonal lattice symmetry. Nanoparticle dimensions can typically be less than about 10 microns, such as less than about 3 microns, less than about 1 micron, less than about 500 nm, less than about 100 nm or less than 50 nm. S-layer proteins can carry, or be linked to, functional domains in a defined position and orientation that enable them to interact with other biomolecules in a highly controlled and well-organized way so that S-layers can be used as carriers for those biomolecules.

After isolation from the cell wall or in the case of recombinant S-layer proteins after extraction out of inclusion bodies, many S-layer proteins maintain the ability to self-assemble in suspension or to recrystallize on solid supports and interfaces (e.g., lipid films, air water interface) with the same repetitive physicochemical properties found originally on the cell, thus forming a stable uniform crystalline monolayer. Crystalline S-layer fusion protein coatings allow for the reproducible, dense, oriented, and uniform presentation of binding sites while at the same time improving signal-to-noise ratios due to the intrinsic anti-fouling properties of the S-layer opening a broad potential for application in biotechnology, molecular nanotechnology and biomimetics.

As used herein, the term “S-layer protein” or a domain thereof encompasses S-layer polypeptides that self-assemble. For example, the term “S-layer protein” explicitly includes polypeptides that are truncated, e.g., C-terminal truncated, as compared to naturally occurring S-layer proteins but which retain the ability to self-assemble. For example, the C-terminal truncated rSbpA₃₁₋₁₀₆₈ is a commonly used molecular building block that is self-assembling.

S-layer proteins are found in bacteria including, but not limited to, Bacillus thuringiensis, Bacillus cereus, Lysinibacillus sphaericus and Geobacillus stearothermophilus. In certain aspects, the S-layer protein is SbpA from Lysinibacillus sphaericus CCM 2177. Wild-type (wt) SbpA protein can be directly extracted and purified from bacteria Lysinibacillus sphaericus (ATCC 4525). The S-layer protein SbpA from Lysinibacillus sphaericus CCM 2177 can induce self-assembly by adding CaCl₂ to a monomeric protein solution. Self-assembly of the wtSbpA with long range ordering can occur on solid surfaces, for example, a pharmaceutically acceptable nanoparticle, and can have a lattice parameter or dimension of about 13 nm. The S-layer protein can also be the S-layer protein from Geobacillus stearothermophilus PV72/p2 (SbsB) or Geobacillus stearothermophilus NRS 2004/3a (SgsE). (Sleytr et al. (2014) FEMS Microbiol. Rev. 38: 823-864 (Table 2) the contents of which are expressly incorporated by reference herein.

In certain aspects, the S-layer protein can be a recombinant protein. Recombinant S-layer proteins can, for example, be genetically-modified and expressed in a production organism, such as E. coli, including truncated self-assembling domains.

In certain embodiments, the S-layer can attach via the N-terminus to a pharmaceutically acceptable nanoparticle with the viral spike protein exposed on the outermost surface of the protein lattice.

SARS-CoV-2 is a β-coronavirus. Other β-coronaviruses include SARS-CoV-1, MERS-CoV, as well as the common cold human CoVs (HCoV-OC43 and HCoV-HKU-1). The spike protein or a fragment thereof, can for example, be recombinantly produced. The preparation of recombinant RBDs from SARS-CoV-2 and other coronaviruses has been described, for example, in Premkumar et al. (2020). The receptor-binding domain of the viral spike protein is an immunodominant and highly specific target of antibodies in SARS-CoV-2 patients. Sci. Immunol. 5, eabc8413; the contents of which are expressly incorporated by reference herein. The genomic sequence of SARS-CoV-2 has been described in Wu et al. (2020). A new coronavirus associated with human respirator disease in China. Nature 579; the contents of which are expressly incorporated by reference herein.

The SARS-CoV-2 spike protein is currently a major focus of vaccine development and it has been shown that an antibody response was elicited in rabbits immunized with S1 domain alone, the RBD, and the S 1+S2 domains together and that S2 alone elicited only a weak response (fda.gov/vaccines-blood-biologics/biologics-research-projects/study-antibody-response-sars-cov-2-spike-proteins-could-help-inform-vaccine-design; the contents of which are expressly incorporated by reference herein). In certain embodiments, the spike protein fragment is an immunogenic fragment. In yet other aspects, the spike protein fragment comprises the S1 domain, the RBD, and/or the RBM. In yet additional aspects, the spike protein or fragment thereof is a coronavirus spike protein or a fragment thereof. Non-limiting examples of coronavirus spike proteins are SARS-CoV-1, SARS-CoV-2, MERS, HCoV-OC43 and HCoV-HKU-1 spike proteins. In preferred aspects, the coronavirus spike protein is the SARS-CoV-2 spike protein or a fragment thereof.

The S-layer fusion protein comprises an S-layer protein and a viral spike protein or a fragment thereof (for example, an immunogenic fragment). Such fusion proteins can comprise the self-assembling S-layer protein and a fused functional viral spike protein sequence or a fragment thereof (collectively referred to herein as the “spike domain” of the fusion protein). The “spike domain” of an S-layer fusion protein can be fused directly or indirectly to the S-layer proteins, for example, via a linker sequence to the S-layer protein. For example, the fusion protein comprising recombinant SbpA (rSbpA) can be constructed using rSbpA in its truncated form which retains its recrystallization property. The spike domain can be fused to an S-layer protein, for example, at the C-terminus of the self-assembling domain of a truncated S-layer protein. In certain aspects of the invention, the S-layer fusion protein is rSbpA₃₁₋₂₀₆₈ZZ (ZZ is the IgG binding domain of Protein A). The N-terminus of the S-layer fusion protein can optionally be bound to the surface of a solid substrate (such as a liposome, or other pharmaceutically acceptable nanoparticle) and, as such, the spike domain is fused to the C-terminus of the S-layer protein. Of course, the reverse configuration is also contemplated.

That S-layer proteins can be fused to foreign proteins or domains while retaining the ability to self-assemble has been described, for example, in Sleytr et al. (2014). The S-layer fusion tag can be linked to the spike protein or fragment thereof through a variety of functional groups and/or ligand binding interactions. As defined herein, an “S-layer protein” encompasses an S-layer protein (e.g., a truncated S-layer protein that can self-assemble) and a fusion domain. The “fusion domain” is a polypeptide that is fused to the S-layer protein, for example, it can be fused directly to the S-layer protein or fused via a linker sequence to the S-layer protein. For example, the fusion protein comprising recombinant SbpA (rSbpA) can be constructed using rSbpA in its truncated form which retains its recrystallization property. A binding moiety with affinity for the fusion domain can be directly or indirectly attached to a spike protein or a fragment thereof. As used herein, the term “spike protein or fragment thereof” and the like encompasses a spike protein or a fragment thereof fused to a binding moiety. The fusion domain can, for example, be streptavidin, an Fc binding region (for example, an Fc binding region from Protein A or the Fc binding region from Protein G), or antibody or antigen, or any other sequence or moiety that has binding affinity for a binding moiety on the spike protein. The fusion domain can be fused to an S-layer protein, for example, a C-terminally truncated S-layer protein. The C-terminally truncated S-layer protein can, for example, be the C-terminally truncated form of rSbpA. An S-layer-streptavidin fusion protein has also been described in Moll (2002), PNAS 99(23):14646-14651. In addition, an exemplary S-layer fusion protein comprising the Fc binding domain of Protein A is the S-layer fusion protein rSbpA.sub.31-1068ZZ incorporating 2 copies of the 58 amino acid Fc-binding Z-domain (a synthetic analogue of the IgG binding domain of protein A from Staphylococcus aureus) (Vollenkle et al. (2004), Appl Environ Microbiol. 2004; 70:1514-1521. Highlight in Nature Reviews Microbiology 1512(1515), 1353 and Ilk et al. (2011), Curr Opin Biotechnol 22(6): 824-831, the contents of each of which are incorporated by reference herein in). Another exemplary S-layer fusion protein is a fusion protein comprising the Fc binding moiety of Protein G and rSbpA (for example, rSbpA GG described, for example, in Ucisik et al. (2015), Colloids Surf B Biointerfaces 128: 132-139). In certain aspects of the invention, the S-layer fusion protein is rSbpA.sub.31-1068ZZ. The fusion domain can, for example, be fused to the C-terminus of the S-layer protein. Additional functional recombinant S-layer fusion proteins have been described in Sleytr et al.

(2014) and includes those shown in Table 1 below:

TABLE 1
Functional recombinant S-layer fusion proteins and their applications
(from Sleytr et al. (2014). FEMS Microbiol Rev 38 (2014) 823-864)

| Recombinant S- | | Length of | | |
|---|---|---|---|---|
| layer protein | Functionality | function | Application | References |
| SbpA SbsB | Core streptavidin | 118 aa | Binding of biotinylated | Moll et al. (2002) |
| | | | ligands (DNA, protein), | and Huber et al. |
| | | | Biochip development | (2006b) |
| SbpA, SbsC | Major birch pollen allergen | 116 aa | Vaccine development, | Breitwieser et al. |
| | (Bet v1) | | treatment for type 1 | (2002) and Ilk et al. |
| | | | allergy | (2002) |
| SbpA | Strep-tag II, Affinity tag for | 9 aa | Biochip development | Ilk et al. (2002) |
| | streptavidin | | | |
| SbpA | ZZ, IgG-binding domain of | 116 aa | Extracorporeal blood | Völlenkle et al. |
| | Protein A | | purification | (2004) |
| SbpA | Enhanced green | 238 aa | Coating of liposomes, | Ilk et al. (2004) |
| | fluorescent protein (EGFP) | | Development of drug and | |
| | | | delivery systems | |
| SbpA | cAb, Heavy chain camel | 117 aa | Diagnostic systems and | Pleschberger et al. |
| | antibody | | sensing layer for label- | (2004) |
| | | | free detection systems | |
| SbpA | Hyperthermophilic enzyme | 263 aa | Immobilized biocatalysts | Tschiggerl et al. |
| | laminarinase (LamA) | | | (2008b) |
| SbpA | Cysteine mutants | 3 aa | Building of nanoparticle | Badelt-Lichtblau |
| | | | arrays | et al. (2009) |
| SbpA, SbsB | Mimotope of an Epstein- | 20 aa | Vaccine development | Tschiggerl et al. |
| | Barr virus (EBV) epitope | | | (2008a) |
| | (F1) | | | |
| SbpA, SbsB | Mycoplasma tuberculosis | 204 aa | Vaccine development | H. Tschiggerl (pers. |
| | antigen (mpt64) | | | commun.) |
| SbpA | IgG-Binding domain of | 110 aa | Downstream processing | Nano-S Inc. (pers. |
| | Protein G | | | commun.) |
| SgsE | Glucose-1-phosphate | 299 aa | Immobilized biocatalysts | Schäffer et al. |
| | thymidylyltransferase | | | (2007) |
| | (RmlA) | | | |
| SgsE | Enhanced cyan fluorescent | 240 aa | pH biosensors in vivo or in | Kainz et al. |
| | protein (ECFP) | | vitro, fluorescent markers | (2010a, b) |
| | | | for drug delivery systems | |
| | Enhanced green | 240 aa | | |
| | fluorescent protein (EGFP) | | | |
| | Yellow fluorescent protein | 240 aa | | |
| | (YFP) | | | |
| | Monomeric red | 225 aa | | |
| | fluorescent protein (RFP1) | | | |
| SbsA | Haemophilus influenzae | 200 aa | Vaccine development | Riedmann et al. |
| | antigen (Omp26) | | | (2003) |
| SlpA | Antigenic poliovirus | 11 aa | Development of mucosal | Avall-Jääskeläinen |
| | epitope (VP1) | | vaccines | et al. (2002) |
| | Human c-myc proto- | 10 aa | | |
| | oncogene | | | |
| SLH-EA1, SLH- | Levansucrase of B. subtilis | 473 aa | Vaccine development | Mesnage et al. |
| Sap | | | | (1999a) |
| SLH-EA1 | Tetanus toxin fragment C | 451 aa | Development of live | Mesnage et al. |
| | of C. tetani (ToxC) | | veterinary vaccines | (1999c) |
| RsaA | Pseudomonas aeruginosa | 12 aa | Vaccine development | Bingle et al. (1997a) |
| | strain K pilin | | | |
| RsaA | IHNV glycoprotein | 184 aa | Development of vaccines | Simon et al. (2001) |
| | | | against hematopoietic | |
| | | | virus infection | |
| RsaA | Beta-1,4-glycanase (Cex) | 485 aa | Immobilized biocatalysts | Duncan et al. (2005) |
| RsaA | IgG-binding domain of | GB1ₓₛ | Development of | Nomellini et al. |
| | Protein G | | immunoactive reagent | (2007) |
| RsaA | Domain 1 of HIV receptor | 81 aa | Anti-HIV microbicide | Nomellini et al. |
| | CD4 | | development | (2010) |
| | MIP1α ligand for HIV | 70 aa | | |
| | coreceptor CCR5 | | | |
| RsaA | His-tag, Affinity tag | 6 aa | Bioremediation of heavy | Patel et al. (2010) |
| | | | metals (Cd) from aqueous | |
| | | | systems, bioreactor | |
| RsaA | Protective coat | 6 aa | Protection against | Patel et al. (2010) |
| | | | antimicrobial peptide in | and de la Fuente- |
| | | | Caulobacter crescentus | Núñez et al. (2012) |

The S-layer proteins used in the fusion proteins described herein can also be selected from SbsB of Geobacillus stearothermophilus PV72/p2, SbpA of Lysinibacillus sphaericus CCM 2177, SbsC of Geobacillus stearothermophilus ATCC 12980, SgsE of Geobacillus stearothermophilus NRS 2004/3a, SbsA of Bacillus stearothermophilus PV72/p6, SlpA of Lactobacillus brevis ATCC 8287, SLH (SLH domain of EA1 or Sap) of Bacillus anthracis, RsaA of Caulobacter crescentus CB15A.

It has been shown that inter- and intra-molecular crosslinking does not alter or abolish the specific function of the fusion partner. This has been demonstrated, for example, using the ZZ_S-layer (rSbpA) fusion protein. (Breitwieser, A., Pum, D., Toca-Herrera, J. L., and Sleytr, U. B. (2016) Magnetic beads functionalized with recombinant S-layer protein exhibit high human IgG-binding and anti-fouling properties. Current Topics in Peptide and Protein Research. 17: 45-55; the contents of which are expressly incorporated by reference herein). Crosslinking was performed with 10 mM DMP (Dimethyl-pimelimidate-dihydrochloride) in 0.1 M Hepes buffer, pH 8 containing 10mM CaCl2 for 90 min. The S-layer protein fusion peptides, self-assembling units or S-layer proteins can be attached to a nanoparticle or other substrate, for example, by contacting the substrate with the self-assembling units/domains followed by crosslinking. In certain aspects, the surface of the substrate (e.g., the nanoparticle) can be first functionalized with the S-layer proteins (or functionalized with an S-layer protein functionalized with a linking group) and then contacted with the spike domain which binds to the S-layer protein (thus forming the self-assembling unit after attachment of the S-layer protein to the surface). Certain S-layer proteins fold into monomers, dimers, tetramers or hexamers which form the crystalline lattice. The S-layer tetramer can have a dimension of about 13 nm² per 2D unit. In certain aspects, the N-terminus of the S-layer protein is attached to the nanoparticle surface and the C-terminus is linked to the spike protein.

The S-layer protein can also be attached to a surface using a bonding agent such as secondary cell wall polymers (SCWP) of prokaryotic microorganisms as described, for example, in U.S. Pat. No. 7,125,707, the contents of which are expressly incorporated by reference herein.

Cross linking can result in increased stability as the cross-linking will occur within the S-layer subunits (inter- and intra-molecular) and in the presence of amino-groups on the surface also between the S-layer protein coating and the substrate. Cross linking can also involve carboxyl groups e.g. activated with carbodiimide (EDC). Cross-linking can be performed after the coating process when the S-layer fusion proteins are in a binding active state; or after the binding of the spike domain.

The ability of S-layer proteins to self-assemble on a variety of surfaces has been described in the art (see, for example, Ilk et al. (2008), Colloids and Surfaces 321: 163-167, U.S. Pat. App. Pub. No. 2004/0137527, and U.S. Pat. No. 7,262,281, the contents of each of which are expressly incorporated by reference herein).

The spike protein or amino acid sequence thereof can be isolated or derived from a naturally occurring virus. Coronavirus (SARS-CoV-2) spike protein is a preferred protein. In one embodiment, the spike protein can comprise subunit 1. Typically, the spike protein or domain or a fragment thereof will comprise the native amino acid sequence. However, variants that retain the spike binding function on mammalian cells can also be used. Typically, a variant of a native spike domain can comprise at least about 95%, 96%, 97%, 98%, 99%, 99.2%, 99.5%, 99.6%, 99.7%, 99.8%, 99.9%, 99.95%, 99.98% or 99.99% sequence identity with a native spike domain sequence. The spike protein or fragment thereof can also be recombinantly produced. A “coronavirus spike protein” is a spike protein having the amino acid sequence of a coronavirus spike protein. Similarly, a SARS-CoV-2 spike protein is a spike protein having the amino acid sequence of a SARS-CoV-2 spike protein.

The invention further contemplates nucleic acid sequences that encode the fusion proteins of the invention. The nucleic acid sequences of each domain can have the sequence of the native sequence for that domain. Alternatively, the sequence can be codon optimized for recombinant expression, for example, in E. coli.

As described above, the nanoparticle can be further coated with a nucleic acid encoding a polypeptide, e.g., an antigen. The term “nucleic acid” includes any compound and/or substance that comprises a polymer of nucleotides (nucleotide monomer). These polymers are also referred to as polynucleotides. Nucleic acids may be or may include, for example, ribonucleic acids (RNAs), deoxyribonucleic acids (DNAs). In certain preferred aspects, the nucleic acid is an mRNA, e.g., an mRNA that encodes an antigenic or immunogenic viral protein, such as viral spike protein, or a fragment thereof. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, mRNA can comprise nucleoside analogs such as analogs having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the mRNA has a length of or greater than about 0.5 kb, 1 kb, 1.5 kb, 2 kb, 2.5 kb, 3 kb, 3.5 kb, 4 kb, 4.5 kb, 5 kb, 6 kb, 7 kb, 8 kb, 9 kb, 10 kb, 11 kb, 12 kb, 13 kb, 14 kb, or 15 kb. In some embodiments, the mRNA comprises unmodified nucleotides. In some embodiments, the mRNA comprises one or more modified nucleotides. The mRNA can be unmodified or mRNA containing one or more modifications that typically enhance stability. In some embodiments, modifications are selected from modified nucleotides, modified sugar phosphate backbones, and 5′ and/or 3′ untranslated region (UTR). In some embodiments, the one or more modified nucleotides comprise pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 4′thiouridine, 4′-thiocytidine, and/or 2-thiocytidine. mRNAs can be synthesized according to any of a variety of known methods. For example, mRNAs can be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. In some embodiments, in vitro synthesized mRNA can be purified before formulation and attachment to the nanoparticle to remove undesirable impurities including various enzymes and other reagents used during mRNA synthesis.

mRNA synthesis can include the addition of a “cap” on the 5′ end, and a “tail” on the 3′ end. The presence of the cap is important in providing resistance to nucleases found in most eukaryotic cells. The presence of a “tail” serves to protect the mRNA from exonuclease degradation. Thus, in some embodiments, mRNAs include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′-5′ inverted triphosphate linkage; and the 7-nitrogen of guanine is then methylated by a methyltransferase. 2′-O-methylation may also occur at the first base and/or second base following the 7-methyl guanosine triphosphate residues. Examples of cap structures include, but are not limited to, m7GpppNp-RNA, m7GpppNmp-RNA and m7GpppNmpNmp-RNA (where m indicates 2′-Omethyl residues). In other aspects, the mRNA includes a 3′ poly(A) tail structure. A poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail. In some embodiments, the mRNA includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length. In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer. In some embodiments, the mRNA comprises a 5′ untranslated region (5′ UTR) and/or a 3′ untranslated region (3′ UTR). Nucleic acids can be codon optimized. A codon-optimized RNA (e.g., mRNA) may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

The nucleic acid can have at least one open reading encoding a protein or polypeptide, including an antigen. In certain aspects, an open reading frame (ORF) is codon optimized, e.g., using optimization algorithms. An “open reading frame” is a continuous stretch of DNA beginning with a start codon (e.g., methionine (ATG)), and ending with a stop codon (e.g., TAA, TAG or TGA) and encodes a polypeptide.

In other aspects, the nucleic acid is an immunostimulatory RNA (isRNA). An isRNA is an RNA that is able to induce an innate immune response. It usually does not have an ORF and thus does not encode an antigen but elicits an immune response by binding to a suitable receptor, e.g., a Toll-like receptor. mRNAs having an ORF can also induce an innate immune response, and thus are also contemplated.

The term “identity” or “sequence identity” is known in the art and refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

The terms “identity”, “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions) times 100). Preferably, the two sequences are of the same length.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

The skilled person will be aware of the fact that different computer programs are available to determine the homology between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

The protein sequences or nucleic acid sequences of the present invention can further be used as a “query sequence” to perform a search against public databases to, for example, to identify other family members or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to protein molecules of the invention. To obtain gapped alignments for comparison purposes, Gapped BLAST can be utilized as described in Altschul et al. (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

Coronavirus strains can be classified by serotype or genotype. Serotype classification involves treatment of the virus with neutralizing antibodies, whereas genotype classification generally involves examining the protein sequence. The spike domain can be derived from SARS-CoV, SARS-CoV-2, and MERS. As SARS-CoV-2 evolves in human patients, spike proteins isolated from such progeny can also be used. A composition of the invention can include one, two, three, four, five or more different spike proteins and fragments thereof and/or spike domains isolated from different genotypes or serotypes. Other viruses that present a spike domain can also be used. For example, the IBV spike protein or domain can be used. In certain embodiments, glycosylated spike proteins (produced in higher cells) are used.

The term “recombinant” as used herein relates to a genome (or RNA sequence, cDNA sequence or protein) having any modifications that do not naturally occur to the corresponding genome (or RNA sequence, cDNA sequence or protein). For instance, a RNA genome (or RNA sequence, cDNA sequence or protein) is considered “recombinant” if it contains an insertion, deletion, inversion, relocation or a point mutation introduced artificially, e.g., by human intervention. Therefore, the RNA genomic sequence (or RNA sequence, cDNA sequence or protein) is not associated with all or a portion of the sequences (or RNA sequence, cDNA sequence or protein) with which it is associated in nature. The term “recombinant” as used with respect to a virus, means a virus produced by artificial manipulation of the viral genome. The term “recombinant virus” encompasses genetically modified viruses.

The present invention also includes the immunogenic compositions or vaccines comprising the fusion protein. The term “vaccine” is intended to embrace a composition that is pharmaceutically acceptable and can induce a protective immunological response in a host such that resistance to new infection will be enhanced and/or the clinical severity of the disease reduced.

In another specific aspect of the immunogenic composition according to the present invention the immunogenic composition comprises a pharmaceutically acceptable carrier. The term “pharmaceutical-acceptable carrier” includes solvents, dispersion media, coatings, stabilizing agents, diluents, preservatives, antibacterial and antifungal agents, isotonic agents, adsorption delaying agents, adjuvants, immune stimulants, and combinations thereof. “Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylenediaminetetracetic acid. Carriers include sucrose gelatin, chitosan, hydrogels, and/or phosphate buffered saline.

Chitosan is a natural deacetylated polysaccharide from chitin in crustaceans (e.g., shrimp, crab), insects, and other invertebrates. Recently, Rauw et al. 2009 (Vet Immunol Immunop 134:249-258) demonstrated that chitosan enhanced the cellular immune response of live Newcastle disease vaccine and promoted its protective effect. Further, Wang et al., 2012 (Arch Virol (2012) 157:1451-1461) have shown results revealing the potential of chitosan as an adjuvant for use in a live attenuated influenza vaccine.

The immunogenic composition can further include one or more other immunomodulatory agents such as, e.g. interleukins, interferons, or other cytokines. The immunogenic composition can also contain an adjuvant. “Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.), John Wiley and Sons, NY, pp 51-94 (1995) and Todd et al., Vaccine 15:564-570 (1997). Exemplary adjuvants are the SPT emulsion described on page 147 of “Vaccine Design, The Subunit and Adjuvant Approach” edited by M. Powell and M. Newman, Plenum Press, 1995, and the emulsion MF59 described on page 183 of this same book.

An adjuvant or nanoparticle carrier can include polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents. The products sold under the name Carbopol; (BF Goodrich, Ohio, USA) are particularly appropriate. They are cross-linked with an allyl sucrose or with allyl pentaerythritol. Among them, there may be mentioned Carbopol 974P, 934P and 971P. Most preferred is the use of Carbopol 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose.

Alternatively, the adjuvant may be at a concentration of about 0.01 to 50%, preferably at a concentration of about 2% to 30%, more preferably at a concentration of about 5% to 25%, still more preferably at a concentration of about 7% to 22%, and most preferably at a concentration of 10% to 20% by volume of the final product. In another specific aspect of the immunogenic composition can be effective in the treatment and/or prophylaxis of clinical signs caused by viral infection in a subject of need.

In another specific aspect of the immunogenic composition can be formulated for a single-dose or multiple doses. The composition can be administered subcutaneously, intramuscularly, oral, in ovo, via spray, via drinking water or by eye drop. The present invention provides a method for immunizing a subject comprising administering to such subject an immunogenic composition as described herein.

The term “immunizing” relates to an active immunization by the administration of an immunogenic composition to a subject to be immunized, thereby causing an immunological response against the antigen included in such immunogenic composition.

Preferably, immunization results in lessening of the incidence of the particular infection in a patient or in the reduction in the severity of clinical signs caused by or associated with the particular virus.

Further, the immunization of a subject in need with the immunogenic compositions as provided herewith, results in preventing infection of a subject. Even more preferably, immunization results in an effective, long-lasting, immunological-response against infection. It will be understood that the said period of time will last more than 1 month, preferably more than 2 months, preferably more than 3 months, more preferably more than 4 months, more preferably more than 5 months, more preferably more than 6 months. It is to be understood that immunization may not be effective in all subjects immunized.

The term “treating or preventing” refers to the lessening of the incidence of the particular infection in a flock or the reduction in the severity of clinical signs caused by or associated with the particular infection. Thus, the term “treating or preventing” also refers to the reduction of the number of subjects in a patient population that become infected with the particular virus (e.g., lessening of the incidence of infection) or to the reduction of the severity of clinical signs normally associated with or caused by infection or the reduction of virus shedding after infection in a group of subjects which subjects have received an effective amount of the immunogenic composition as provided herein in comparison to a group of subjects which subjects have not received such immunogenic composition.

The “treating or preventing” generally involves the administration of an effective amount of the immunogenic composition of the present invention to a subject or group of subjects in need of or that could benefit from such a treatment/prophylaxis. The term “treatment” refers to the administration of the effective amount of the immunogenic composition once the subject or at least some subjects of a cohort is/are already infected and wherein such subjects already show some clinical signs caused by or associated with such infection. The term “prophylaxis” refers to the administration of a subject prior to any infection of such subject or at least where such subject or none of the subjects in a group of subjects do not show any clinical signs caused by or associated with the infection. The terms “prophylaxis” and “preventing” are used interchangeable in this application.

The term “an effective amount” as used herein means, but is not limited to an amount of the spike protein, that elicits or is able to elicit an immune response in a subject. Such effective amount is able to lessen the incidence of infection in a patient cohort or to reduce the severity of clinical signs of infection.

Preferably, clinical signs are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, still more preferably by at least 95% and most preferably by 100% in comparison to subjects that are either not treated or treated with an immunogenic composition that was available prior to the present invention but subsequently infected.

The term “clinical signs” as used herein refers to signs of infection of a subject. The clinical signs of infection depend on the pathogen selected. Examples for such clinical signs include but are not limited to respiratory distress.

The term “in need” or “of need”, as used herein means that the administration or treatment is associated with the boosting or improvement in health or clinical signs or any other positive medicinal effect on health of the subjects which receive the immunogenic composition in accordance with the present invention.

The term “reducing” or “reduced” or “reduction” or lower” are used interchangeable in this application. The term “reduction” means, that the clinical sign is reduced by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, even more preferably by at least 90%, still more preferably by at least 95% most preferably by 100% in comparison to subjects that are not treated (not immunized) but subsequently infected.

While this invention has been particularly shown and described with references to preferred embodiments thereof, it will be understood by those skilled in the art that various changes in form and details may be made therein without departing from the scope of the invention encompassed by the appended claims.

1. An S-layer fusion protein comprising a self-assembling domain of an S-layer protein and a viral spike protein or a fragment thereof.

2. The fusion protein of claim 1, wherein the viral spike protein comprises the amino acid sequence of a native coronavirus spike protein.

3. The fusion protein of claim 1, wherein the viral spike protein comprises the amino acid sequence of a native SARS-CoV-2 spike protein.

4. The fusion protein of claim 1, wherein the fragment thereof is an immunogenic fragment.

5. The fusion protein of claim 1, wherein the fragment comprises the S1 domain.

6. The fusion protein of claim 1, wherein the fragment comprises the receptor binding domain (RBD).

7. The fusion protein of claim 1, wherein the fragment comprises the receptor binding motif (RBM).

8. The fusion protein of claim 1, wherein the self-assembling domain comprises truncated rSbpA31-1068 (from Lysinibacillus sphaericus CCM 2177).

9. The fusion protein of claim 1, wherein the self-assembling domain is an S-layer protein from a mesophilic or thermophilic organism.

10. The fusion protein of claim 1, wherein the self-assembling domain comprises (truncated) rSbsB of Geobacillus stearothermophilus PV72/p2, SbsC of Geobacillus stearothermophilus ATCC 12980, SgsE of Geobacillus stearothermophilus NRS 2004/3a, SbsA of Bacillus stearothermophilus PV72/p6, SlpA of Lactobacillus brevis ATCC 8287, SLH (SLH domain of EA1 or Sap) of Bacillus anthracis, RsaA of Caulobacter crescentus CB15A.

11. The fusion protein of claim 1, wherein the viral spike protein is bound to the self-assembling domain via an amino acid linker sequence.

12. The fusion protein of claim 1, wherein the C-terminus of the self-assembling domain is linked to the spike protein.

13. A pharmaceutical composition comprising an effective amount of the S-layer fusion protein of claim 1 and a pharmaceutically acceptable carrier.

14. The pharmaceutical composition of claim 13, wherein the composition is a vaccine.

15. The pharmaceutical composition of claim 14, wherein the vaccine is a mucosal vaccine.

16. The vaccine of claim 15, for intranasal or oral administration.

17. A method of immunizing a patient in need thereof comprising administering to the patient the vaccine of claim 14.

18. The method of claim 17, wherein the vaccine is administered intranasally or orally.

19. The method of claim 17, wherein the spike protein or the fragment thereof is a coronavirus spike protein or an immunogenic fragment thereof and the patient is immunized against a coronavirus infection.

20. The method of claim 19, wherein the spike protein or the fragment thereof is a SARS-CoV-2 spike protein or immunogenic fragment thereof and the patient is immunized against COVID-19.

21. A composition comprising a plurality of S-layer fusion proteins, wherein the S-layer fusion protein comprises a self-assembling domain of a S-layer protein and a viral spike protein or a fragment thereof, and wherein the plurality of S-layer fusion proteins form a self-assembled structure.

22. The composition of claim 20, wherein the self-assembled structure is selected from the group consisting of a flat sheet, an open-ended cylinder, and a vesicle.

23. The composition of claim 20, wherein the self-assembled structure is a monolayer.

24. The composition of claim 20, wherein the self-assembled structure is a double layer.

25. The composition of claim 21, wherein the composition comprises an effective amount of the S-layer fusion protein or the fragment thereof and wherein the composition further comprising a pharmaceutically acceptable carrier.

26. The pharmaceutical composition of claim 25, wherein the composition is a vaccine.

27. The pharmaceutical composition of claim 26, wherein the vaccine is a mucosal vaccine.

28. The vaccine of claim 27, for intranasal or oral administration.

29. A method of immunizing a patient in need thereof comprising administering to the patient the vaccine of claim 26.

30. The method of claim 29, wherein the vaccine is administered intranasally or orally.

31. The method of claim 29, wherein the spike protein or the fragment thereof is a coronavirus spike protein or an immunogenic fragment thereof and the patient is immunized against a coronavirus infection.

32. The method of claim 31, wherein the spike protein or the fragment thereof is a SARS-CoV-2 spike protein or immunogenic fragment thereof and the patient is immunized against COVID-19.

33. A nanoparticle coated with a plurality of S-layer fusion proteins, wherein the S-layer fusion protein comprises a self-assembling domain of a S-layer protein and a viral spike protein or a fragment thereof,
wherein the self-assembling domain is attached to the surface of the nanoparticle, and wherein the plurality of S-layer fusion proteins form a crystalline lattice on the surface of the nanoparticle.

34. The nanoparticle of claim 33, wherein the N-terminus of the self-assembling domain of the S-layer protein is directly or indirectly attached to the surface of the nanoparticle.

35. The nanoparticle of claim 33, wherein the nanoparticle is a lipid vesicle.

36. The nanoparticle of claim 35, wherein the lipid vesicle is a liposome.

37. The nanoparticle of claim 36, wherein the liposome encapsulates a liphophilic or a hydrophilic compound.

38. The nanoparticle of claim 33, wherein the plurality of S-layer fusion proteins comprises a first population of S-layer fusion proteins and a second population of S-layer fusion proteins, wherein the viral spike protein or fragment thereof of the first population is different from the viral spike protein or fragment thereof of the second population.

39. A pharmaceutical composition comprising an effective amount of the nanoparticle of claim 33 and a pharmaceutically acceptable carrier.

40. The pharmaceutical composition of claim 39, wherein the composition is a vaccine.

41. The pharmaceutical composition of claim 40, wherein the vaccine is a mucosal vaccine.

42. The vaccine of claim 41, for intranasal or oral administration.

43. A method of immunizing a patient in need thereof comprising administering to the patient the vaccine of claim 40.

44. The method of claim 43, wherein the vaccine is administered intranasally or orally.

45. The method of claim 43, wherein the spike protein or fragment thereof is a coronavirus spike protein or an immunogenic fragment thereof and the patient is immunized against a coronavirus infection.

46. The method of claim 45, wherein the spike protein or fragment thereof is a SARS-CoV-2 spike protein or immunogenic fragment thereof and the patient is immunized against COVID-19.

47. An isolated inclusion body comprising the fusion protein of claim 1, wherein the inclusion body is a particle.

48. A pharmaceutical composition comprising an effective amount of the inclusion body of claim 47 and a pharmaceutically acceptable carrier.

49. The pharmaceutical composition of claim 48, wherein the composition is a vaccine.

50. The pharmaceutical composition of claim 48, wherein the vaccine is a mucosal vaccine.

51. The pharmaceutical composition of claim 50, wherein the inclusion body is freeze dried.

52. A method of immunizing a patient in need thereof comprising administering to the patient the vaccine of claim 49.

53. The method of claim 52, wherein the vaccine is administered intranasally or orally.

54. The method of claim 52, wherein the spike protein or a fragment thereof is a coronavirus spike protein or immunogenic fragment thereof and the patient is immunized against a coronavirus infection.

55. The method of claim 54, wherein the spike protein is a SARS-CoV-2 spike protein or immunogenic fragment thereof and the patient is immunized against COVID-19.

56. The method of claim 54, wherein the vaccine is administered more than once.
